(19) United States
(12) Patent Application Publication
(10) Pub. No.: US 2024/0221898 A1
(43) Pub. Date: Jul. 4, 2024

(54) DIALYSIS MACHINE WITH CONSUMABLES MANAGEMENT

(71) Applicant: CVS PHARMACY, INC.

(72) Inventors: Burak Say, Burlington, MA (US); Daniel Carleton, Indianapolis, IN (US); Howard Cardoza, Cranston, RI (US); Derek Wiebenson, Lafayette, CO (US); Troy Isenhart, Boston, MA (US)

(21) Appl. No.: 18/397,794

(22) Filed: Dec. 27, 2023

Related U.S. Application Data
(60) Provisional application No. 63/435,988, filed on Dec. 29, 2022.

(51) Int. Cl. G16H 20/40; A61M 1/16; G16H 40/63

(52) U.S. Cl. CPC G16H 20/40; A61M 1/1603; G16H 40/63; A61M 2205/52

(57) ABSTRACT

A method of providing home dialysis treatment with a dialysis machine includes performing, by the dialysis machine, dialysis on a patient using a first unit of a consumable, providing, by the dialysis machine as a result of performing the dialysis, operating data indicating consumption of the first unit, estimating a remaining count of the consumable based on the operating data, forecasting future demand for the consumable based on the operating data, providing a quantity of new units of the consumable to the dialysis machine based on a comparison of the forecasted future demand and the estimated remaining count, and performing, by the dialysis machine dialysis on the patient using one or more of the new units of the consumable.

### CROSS-REFERENCE TO RELATED APPLICATION

This application claims priority to and the benefit of U.S. Provisional Patent Application No. 63/435,988 filed Dec. 29, 2022, the entire disclosure of which incorporated by reference herein in its entirety.

### BACKGROUND

The present disclosure relates to systems and methods for managing consumables associated with a medical device. In particular, according to some examples, the present disclosure relates to nephrology, in particular dialysis treatment (e.g., hemodialysis, peritoneal dialysis) for patients with kidney disorders, dysfunction, or other conditions which can be managed through dialysis treatment. In hemodialysis treatment, for example, a patient's blood is removed from a patient and provided to a dialysis machine. The dialysis machine filters waste from the blood and provides the filtered blood back to the patient. The dialysis machine thereby operates as an artificial kidney.

New technologies which enable at-home dialysis can improve patients' access to dialysis, for example facilitating more frequent treatments and providing other advantages which may improve clinical outcomes (e.g., as compared to a conventional approach of providing dialysis machines at dedicated treatment centers to which patients travel for dialysis treatment). One technical barrier to at-home dialysis technologies is that a variety of consumables (e.g., supplies, tools, instruments, dialysate, prescribed compounds, etc.) are consumed (e.g., used and discarded or used up) during operation of a dialysis machine to provide dialysis, without which an at-home dialysis machine cannot properly operate.

### SUMMARY

One implementation of the present disclosure is a dialysis system. The dialysis system includes a dialysis machine configured to perform dialysis to a patient while causing consumption of a consumable. The dialysis system also includes circuitry programmed to automatically cause additional units of the consumable to be provided to the dialysis machine in a quantity determined, by the circuitry, based on estimated historical consumption of the consumable and an estimated future consumption of the consumable.

In some examples, the circuitry is programmed to determine the quantity further based on an order history for the consumable. In some examples, the circuitry is further programmed to provide a graphical user interface comprising an option for a user to adjust the quantity.

Another implementation of the present disclosure is a method of automatically providing supplies for home dialysis treatment with a dialysis machine. The method includes receiving, from the dialysis machine, operating data indicating consumption of a first unit of a consumable in conjunction with the home dialysis treatment, estimating a remaining amount of the consumable based on the operating data, forecasting future demand for the consumable based on the operating data, and causing a quantity of new units of the consumable to be delivered to a location of the dialysis machine based on a comparison of the forecasted future demand and the estimated remaining amount to be used for subsequent home dialysis treatments.

In some examples, estimating the remaining amount of the consumable based on the operating data includes determining, from the operating data, a number of treatment sessions provided by the dialysis machine and estimating a historical consumption of the consumable based on the number of treatment sessions.

In some examples, the method also includes providing a user interface that includes an indication of the remaining amount of the consumable or the quantity of new units of the consumable. In some examples, the method also includes allowing a user to adjust the quantity of new units of the consumable via the user interface. In some examples, estimating the remaining amount of the consumable is also based on an order history for the consumable.

Another implementation of the present disclosure is non-transitory computer-readable media storing program instructions that, when executed by the one or more processors, cause the one or more processors to perform operations. The operations can include receiving, from a dialysis machine, operating data indicating consumption of a first unit of a consumable in conjunction with the home dialysis treatment, estimating a remaining amount of the consumable based on the operating data, forecasting future demand for the consumable based on the operating data, and causing a quantity of new units of the consumable to be delivered to a location of the dialysis machine based on a comparison of the forecasted future demand and the estimated remaining amount to be used for subsequent home dialysis treatments.

In some examples, the operations also include providing a user interface comprising an indication of the remaining amount of the consumable or the quantity of new units of the consumable. In some examples, the operations also include enabling a user to adjust the quantity of new units of the consumable via the user interface. In some examples, estimating the remaining amount of the consumable is further based on an order history for the consumable.

This summary is illustrative only and should not be regarded as limiting. All examples and features mentioned above can be combined in any technically possible way.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a diagram of a dialysis machine and consumables for use with the dialysis machine, according to some examples.

FIG. 2 is an illustration of a dialysis system including the dialysis machine, according to some examples.

FIG. 3 is a flowchart of a process for operating the dialysis system, according to some examples.

FIG. 4 is another flowchart of a process for operating the dialysis system, according to some examples.

FIG. 5 is an illustration of a user device displaying an interface relating to dialysis treatment, according to some examples.

FIG. 6 is an illustration of a user device displaying another interface relating to dialysis treatment, according to some examples.

FIG. 7 is an illustration of a user device displaying yet another interface relating to dialysis treatment, according to some examples.

FIG. 8 is an illustration of a user device displaying yet another interface relating to dialysis treatment, according to some examples.

FIG. 9 is an illustration showing a sequence of interfaces display on a user device, according to some examples.

### DETAILED DESCRIPTION

Referring generally to the figures, systems and methods for helping ensure a dialysis system has a supply of consumables to conduct dialysis treatments is shown, according to some examples. The dialysis-related systems of the present disclosure are adapted for at-home treatment, i.e., for use in providing dialysis to patients at locations other than a dedicated dialysis center, clinic, hospital, etc., for example at a patient's home (residence, etc.). The dialysis systems herein are configured to provide automated management of consumable inventories to ensure that suitable consumables are available at a dialysis machine (e.g., at a patient's home) to enable successful dialysis treatment of a patient by the dialysis machine. In some examples, operating data from a dialysis machine is used to determine quantities of various consumables to provide for use with the dialysis machine over time. The teachings herein can avoid both oversupplying consumables (leading to waste, storage issues, lapsing of expiration dates, overspending) and undersupplying consumables (potentially leading to missed treatments for a patient), thereby improving overall operation of at-home dialysis machines and execution of at-home dialysis treatment. It should be understood that, as utilized herein, the provision of automated management of consumable inventories includes both fully automated management in which the system provides quantities of consumables without human confirmation/interaction and human-in-the-loop/human-on-the-loop management in which the system automatically determines proposed quantities but requires or allows for human (e.g., patient and/or physician) confirmation of the proposed quantities and/or allows for overriding/modification by a human of the proposed quantities.

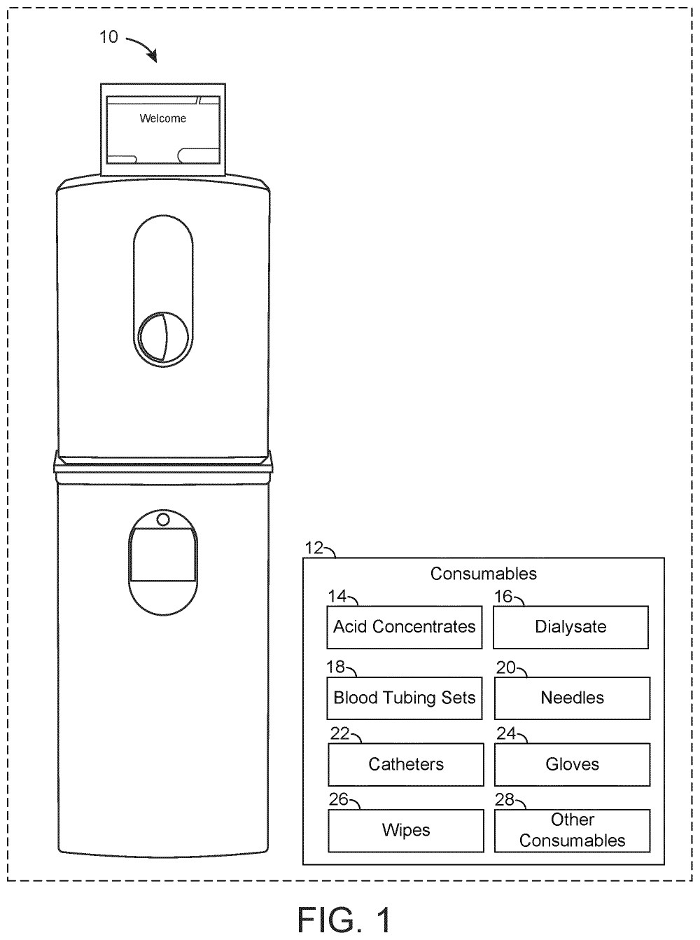

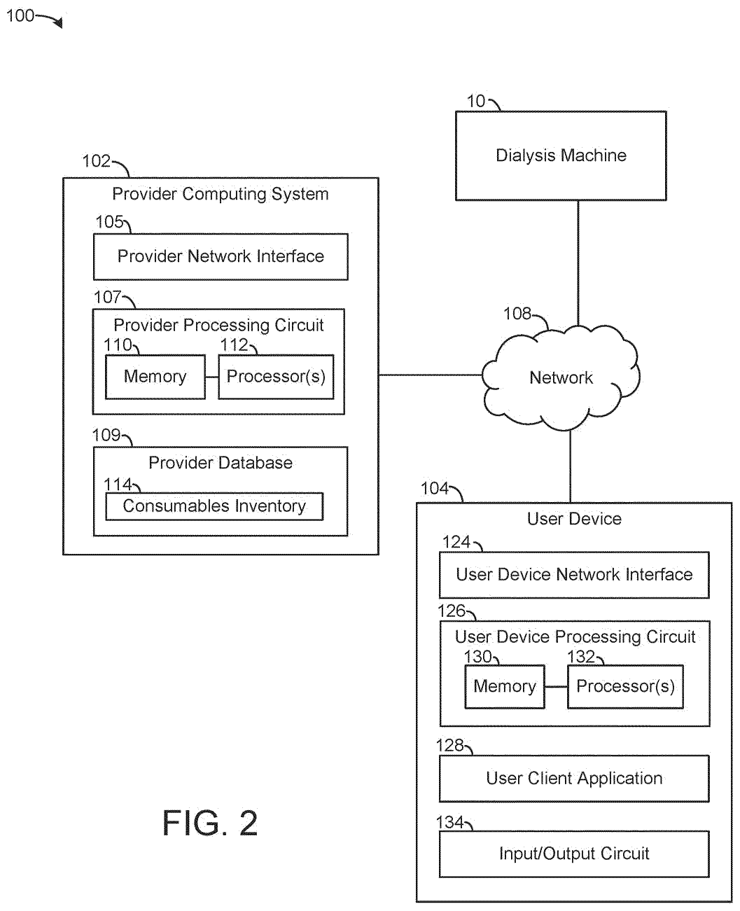

Referring now to FIG. 1, an illustration of a dialysis machine 10 and consumables 12 is shown, according to some examples. The dialysis machine 10 and the consumables 12 can be elements of a dialysis system, for example dialysis system 100 of FIG. 2 (which is described in further detail below). In the example shown, the consumables 12 include acid concentrates 14, dialysate 16, blood tubing sets 18, needles 20, catheters 22, gloves 24, wipes 26, and various other consumables 28 (e.g., accessories, instruments, compounds, drugs, fluids, tools, etc.). It should be understood that these are only examples of consumables that may be utilized in conjunction with the present disclosure; in various implementations, other types of consumables may be used. The dialysis machine 10 is configured to provide dialysis treatment to a patient, and operating the dialysis machine 10 to provide dialysis treatment involves consumption of one or more of the consumables 12.

Some consumables 12 are directly consumed by the dialysis machine 10. For example, acid concentrates 14 and dialysate 16 are provided into the dialysis machine 10 and used by the dialysis machine 10 in treating (e.g., filtering, cleaning) a patient's blood. In some examples, the dialysis machine 10 is configured to track (e.g., measure) quantities of such consumables 12 used by the dialysis machine 10.

As another example, blood tubing sets 18 can be coupled to the dialysis machine 10 in order to provide a path for blood to flow from the patient to the dialysis machine 10 and from the dialysis machine 10 to the patient. Each blood tubing set 18 may be suitable for use in a finite number of treatments, and can thus be considered as being consumed by operation of the dialysis machine 10. In some examples, the dialysis machine 10 includes a sensor, receiver, barcode scanner, etc. configured to detect when a blood tubing set 18 is removed from the dialysis machine 10 and/or when a new blood tubing set 18 is provided for use with the dialysis machine 10. The dialysis machine 10 can thereby track consumption of blood tubing sets 18, in some examples.

Some consumables 12 are single-use tools, instruments, accessories, etc. which are intended to be discarded after use (e.g., for sterilization reasons). For example, for some patient treatments, a set number of one or more needles 20 and/or one or more catheters 22 are needed per treatment (e.g., one needle 20 per treatment, one catheter 22 per treatment) and such needle(s) 20 and/catheter(s) 22 are then discarded after use. Needles 20 and catheters 22 can thus be considered as being consumed by operation of the dialysis machine 10. Other consumables such as gloves 24 and wipes 26 (e.g., sterilizing wipes) may have a similar relationship with operation of the dialysis machine 10, where gloves 24 and wipes 26 are used and discarded during each dialysis treatment provided by operation of the dialysis machine 10. The gloves 24 and wipes 26 can thus be considered as being consumed by operation of the dialysis machine 10.

As shown in FIG. 1, the consumables 12 are collocated with the dialysis machine 10. For example, both the dialysis machine 10 and the consumables 12 may be located at a patient's home, thereby enabling the patient to conduct at-home dialysis treatments using the dialysis machine 10 and the consumables 12. However, only a finite quantity of the consumables 12 can be located at a patient's home at any given time, given storage constraints.

The various consumables 12 are likely to be consumed at variable rates which depends on a number of treatments a patient undergoes (e.g., twice per day, once per day, every-other day, etc.), configuration of such treatments (e.g., duration of each treatment, prescribed compounds/fluids to be used in each treatment, operating settings of the dialysis machine 10), user preferences and/or ability (e.g., preference for use of multiple sterilization wipes, use or non-use of gloves for various steps of a treatment, success rate at inserting needles and catheters, etc.), and/or other conditions or variables.

As use of the dialysis machine results in consumption of the consumables 12, the available consumables 12 will be depleted and run out, however, due to complexities in the rates of consumption, patients would likely have difficulty in pre-ordering a suitable amount of each of the numerous consumables 12. As described in detail below, the teachings herein provide a dialysis system that causes additional consumables 12 to be provided to the user's home (i.e., to the location of the dialysis machine 10) in a manner which ensures suitable quantities of such consumables 12 remain available to the patient and the dialysis machine 10 over time. A prescribed treatment schedule, and proper operation of the dialysis machine 10, can thus be reliably maintained for the patient.

In other examples, the dialysis machine 10 is replaced by a different type of medical device, for example a medical device adapted to provide a different type of at-home therapy. For example, a machine configured to provide radiation therapy, chemotherapy, asthma treatment or other breathing treatment, treatment of a bodily fluid, diabetes treatment, advanced wound therapy, or other type of medical treatment can be included in place of the dialysis machine 10 in various examples, and the teachings herein can be adapted as appropriate for such medical devices.

Referring now to FIG. 2, a dialysis system 100 is shown, according to some examples. As shown in FIG. 2, the dialysis system 100 includes a provider computing system 102, a user device 104, and the dialysis machine 10 communicable via a network 108. In some examples, any number of additional user devices 104 and dialysis machines 10 can be included. The network 108 can be or include the Internet, an intranet, a cellular network, a WiFi network, cloud computing infrastructure, etc., in various examples. In some examples, the provider computing system 102 is physically remote from the user device 104 and/or the dialysis machine 10. The present disclosure contemplates implementations that omits one or two of the dialysis machine 10, the provider computing system 102, and the user device 104, in various examples.

The provider computing system 102 is shown as including a provider network interface 105, a provider processing circuit 107, and a provider database 109. The provider network interface 105 facilitates connection of the provider computing system 102 to the network 108. The network interface 105 can support communication via the network 108 between the user device 104 and the provider computing system 102 and/or between the dialysis machine 10 and the provider computing system 102. The network interface 105 may include communications ports (e.g., Ethernet ports), routing capabilities, a cellular modem, a wireless transceiver or beacon, etc. in various examples. In some examples, the network interface 105 includes cryptographic capabilities to establish a secure communications session.

The provider processing circuit 107 is structured to control, at least partly, the provider computing system 102 and to execute or otherwise enable the various operations attributed herein to the processing circuit 107. For example, the provider processing circuit 107 can execute the various processes shown in the figures and described in detail below, in various examples. The processing circuit includes memory (one or more non-transitory computer readable media) 110 and one or more processors 112. The processor(s) 112 may be implemented as one or more general-purpose processors, application specific integrated circuits (ASICs), field programmable gate arrays (FPGAs), digital signal processors (DSPs), other suitable electronic processing components, or a combination thereof. The memory 110 may be implemented as RAM, ROM, NVRAM, Flash Memory, hard disk storage, solid state storage, etc. and may store data and/or computer-readable instructions (programming, logic, code) for providing the features described herein. The memory 110 stores computer-readable instructions that, when executed by the processor(s) 112, causes the processor(s) 112 to perform some or all of the operations attributed herein to the provider processing circuit 107 and/or provider computing system 102, in various examples.

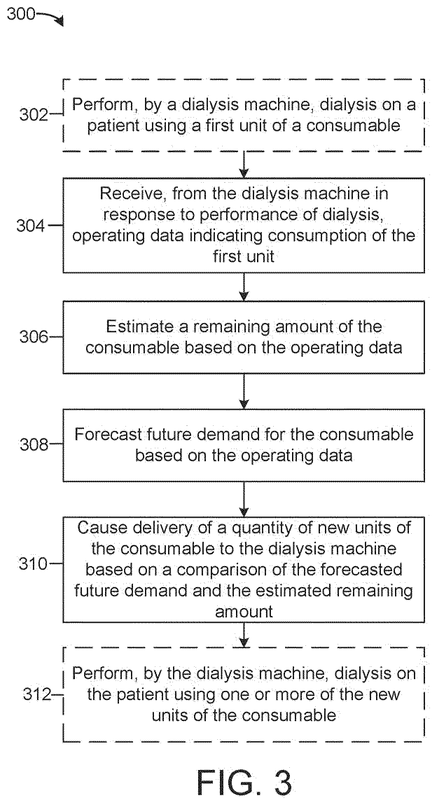

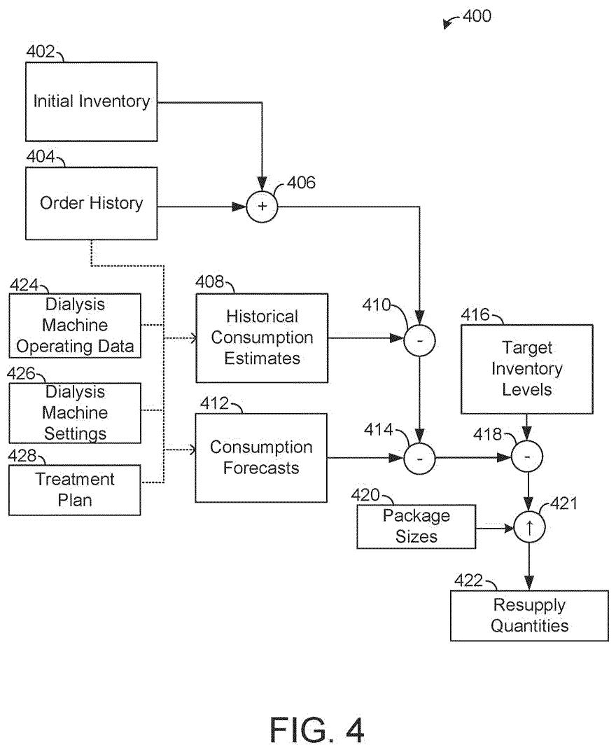

The provider database 109 is structured to retrievably store (e.g., in non-transitory computer memory) data usable by the provider processing circuit 107 for providing operations as described herein. For example, the provider processing circuit 107 may be structured to read data from the provider database 109 and write data to the provider database 109. The provider database 109 can include various database components in various examples, for example memory in hard drive storage, disk storage, solid state storage, etc. FIG. 2 shows the provider database 109 as including consumables inventory 114, which can include an up-to-date estimate of quantities of consumables 12 located at the dialysis machine 10, for example. The consumables inventory 114 can be updated by the provider processing circuit 107, for example according to processes described in detail below with reference to FIGS. 3-4.

The user device 104 as shown in FIG. 2 can be a personal computing device, for example a smartphone, tablet, desktop computer, laptop computer, virtual reality headset, augmented reality headset, etc. In some examples, the user device 104 is a component of the dialysis machine 10. As shown in FIG. 2, the user device 104 includes a user device network interface 124. The user device network interface 124 facilitates connection of the user device 104 to the network 108. The user device network interface 124 can support communication via the network 108 between the user device 104 and the provider computing system 102 and/or between the user device 104 and the dialysis machine 10. The user device network interface 124 may include communications ports (e.g., Ethernet ports), routing capabilities, a cellular modem, a wireless transceiver or beacon (e.g., Bluetooth, near-field communication), etc. in various examples. In some examples, the user device network interface 124 includes cryptographic capabilities to establish a secure communications session.

The user device processing circuit 126 is structured to control, at least partly, the user device 104 and to execute or otherwise enable the various operations attributed herein to the user device 104. For example, the user device processing circuit 126 can execute the various processes shown in the figures and described in detail below, in various examples. The user device processing circuit 126 includes memory (one or more non-transitory computer readable media) 130 and one or more processors 132. The processor(s) 132 may be implemented as one or more general-purpose processors, application specific integrated circuits (ASICs), field programmable gate arrays (FPGAs), digital signal processors (DSPs), other suitable electronic processing components, or a combination thereof. The memory 130 may be implemented as RAM, ROM, NVRAM, Flash Memory, hard disk storage, solid state storage, etc. and may store data and/or computer-readable instructions (programming, logic, code) for providing the features described herein. The memory 130 stores computer-readable instructions that, when executed by the processor(s) 132, causes the processor(s) 112 to perform some or all of the operations attributed herein to the user device processing circuit 126 and/or user device 104, in various examples.

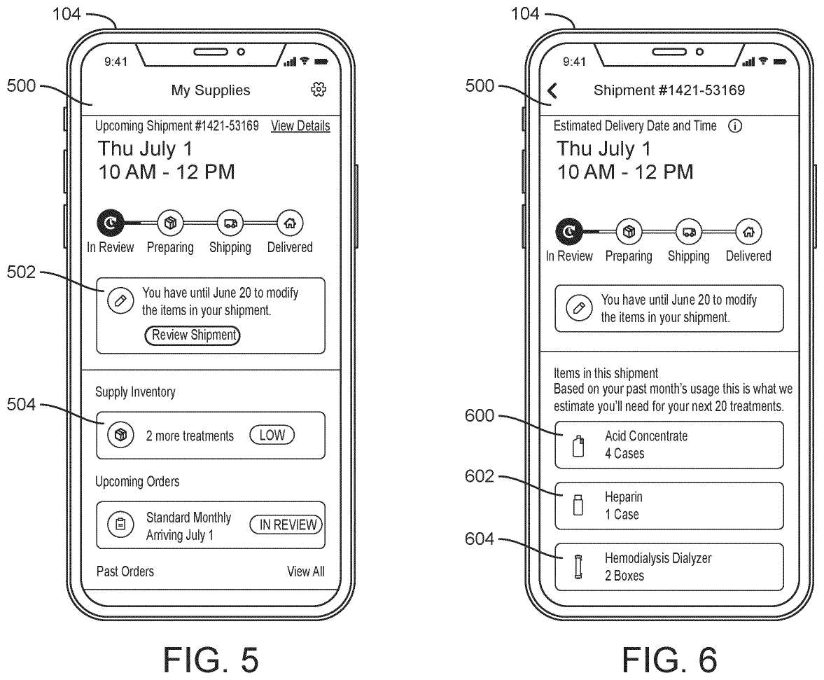

FIG. 2 shows the user device 104 as including a user client application 128. In some examples, the user client application 128 is provided within or by the user device processing circuit 126 (e.g., via instructions stored in memory 130 and executed by processor(s) 132). The user client application 128 can include application code, data, application programming interface(s), etc. to enable use of the user device 104 to access and interoperate with the provider computing system 102. For example, the user client application 128 can be provided as a mobile application on the user device 104, with the mobile application dedicated to enabling features described herein, for example providing interfaces and functionality as shown in FIGS. 5-9 and described in detail below with reference thereto. In some examples, the user device 104 includes a web browser which provides an alternative or additional manner for the user device 104 to interoperate with the provider computing system 102.

The user device 104 is further shown as including an input/output circuit 134. The input/output circuit 134 can include various components for providing outputs to a user of the user device 104 and receiving inputs from a user of the user device 104. For example, the input/output circuit 134 can include a display screen, a touchscreen, a mouse, a button, a keyboard, a microphone, a speaker, an accelerometer, actuators (e.g., vibration motors), including any combination thereof, in various examples. The input/output circuit 134 may also include circuitry/programming/etc. for running such components. The input/output circuit 134 thereby enables communications to and from a user, for example communications relating to consumables 12 as described in further detail elsewhere herein.

FIG. 2 shows the dialysis machine 10 communicable with the network 108. The dialysis machine 10 can include a network interface, for example similar to the user device network interface 124, and a processing circuit, for example configured in a similar manner as described for the user device processing circuit 126. The dialysis machine 10 and circuitry can be configured (e.g., structured, programmed, etc.) to collect operating data relating to operation of the dialysis machine 10 in providing dialysis treatments to a patient. Such operating data can include an indication of each treatment being performed; a duration of each treatment; settings used for each treatment; a number of treatments over a time period; amounts of consumables 12 measured by the dialysis machine 10; indications that consumables 12 were attached, detached, used, removed, discarded, etc.; and/or other data relating to operation of the dialysis machine 10. Such operating data can be made available by the dialysis machine to the provider computing system 102 and/or the user device 104 via the network 108.

Referring now to FIG. 3, a flowchart of a process 300 for a dialysis system (e.g., dialysis system 100) is shown, according to some examples. The process 300 can be executed by or using the dialysis system 100, for example by or using a combination of the dialysis machine 10, the provider computing system 102, and the user device 104, according to some examples. For example, steps 304, 306, and 308 can be performed by the provider processing circuit 107, the user device processing circuit 126, or locally on the dialysis machine 10, or some combination thereof, in various examples.

At step 302, dialysis is performed on a patient by the dialysis machine 10 using a first unit of a consumable 12 (e.g., a unit of acid concentrate 14, a unit of dialysate 16, a blood tubing set 18, a needle 20, a catheter 22, a pair of gloves 24, a wipe 26, some other consumable 28). The patient thereby receives a dialysis, for example in which the patient's blood is filtered, cleaned, etc. and returned to the patient's body. During performance of step 302, the first unit of the consumable is consumed, i.e., rendered into a state where it should not be reused as a result of step 302. The first unit of the consumable may be completely consumed after one treatment session performed by the dialysis machine, or may be consumed by a combination of multiple treatment sessions performed by the dialysis machine. Step 302 can be omitted in some examples.

At step 304, operating data indicating consumption of the first unit of the consumable is received from the dialysis machine 10 (e.g., by the provider computing system 102 and/or the user device 104). In some examples, the dialysis machine 10 self-monitors its operation and generate a report, file, data object, etc. indicating characteristics, attributes, values, etc. describing operation of the dialysis machine in step 302. Operating data collected by and provided by the dialysis machine 10 and indicating consumption of one or more consumables can include an indication that a treatment session was performed, a duration of the treatment session, a time of day and/or date of the treatment session, settings of the dialysis machine 10 during the treatment sessions, measurements made by the dialysis machine 10 during the treatment session, indication of one or more consumables detected by the dialysis machine 10 (e.g., scanned by the dialysis machine 10), etc. In step 304, the operating data can be provided from the dialysis machine 10 to the provider computing system 102 and/or the user device 104, in various examples.

At step 306, a remaining supply of the consumable is estimated (e.g., determined, calculated, approximated, precisely determined) based on the operating data. A number of units of the consumable remaining after consumption of the first unit is estimated in step 306. Step 306 can include determining that the operating data from the dialysis machine 10 indicates that the first unit of the consumable was consumed in step 302 and then subtracting (removing, deleting, etc.) the first unit from a digital inventory of consumables. In some examples, the provider database 109 includes the consumables inventory 114, which indicates numbers of units of one or more types of consumables believed to have been (e.g., calculated to be, estimated to be) located at the dialysis machine 10 prior to performance of dialysis in step 302. In such examples, step 306 includes updating the consumables inventory 114 to reflect that the first unit of the consumable has been consumed (e.g., by reducing a number of the consumable indicated in the consumables inventory 114 by one). FIG. 4 provides further illustration of such a step and is described in detail below. As a result of step 306, an estimated remaining supply of a consumable (e.g., an estimated number of needles 20, a number of catheters 22, etc.) located at the dialysis machine 10 (e.g., at a patient's home where the dialysis machine 10 is located) is determined.

At step 308, future demand for the consumable is forecast, for example based on the operating data from the dialysis machine 10. Step 308 can include determining a number of units of the consumable expected to be consumed over an upcoming time period (e.g., over an upcoming two-week period, over an upcoming month, a user-selectable amount of time, etc.). Step 308 can include using the operating data to inform such forecasts, for example using a model fit to or trained on such operating data (e.g., a regression model fit to a history of past consumption). In some examples, step 308 can include forecasting that future demand for the consumable over an upcoming time period will be substantially the same as over a historical time period of the same duration, for example with historical consumption indicated by the operating data from the dialysis machine 10. In some such examples, the forecasted demand can be adjusted based on any changes to a treatment regimen for the patient or changes to settings to the dialysis machine 10 (e.g., increasing a forecasted number of consumables in response to information indicating that a patient is to increase a frequency of dialysis treatments). Various forecasts, models, input data, etc. are described in further detail below with reference to FIG. 4.

At step 310, a quantity of new units of the consumable is delivered to the dialysis machine 10 (e.g., to the patient's home where the dialysis machine 10 is located) based on a comparison of the forecasted future demand for the consumable and the estimated remaining supply of the consumable. The difference between the forecasted future demand and the estimated remaining supply is an expected number of units of the consumable that would need to be added to a patient's supply (i.e., provided at the dialysis machine 10) for an upcoming time period in order for the dialysis machine 10 to have sufficient consumables available to provide treatment over the upcoming time period. Accordingly, step 310 can include delivering an amount of the consumable to the dialysis machine 10 which is based on the difference between the forecasted future demand and the estimated remaining supply. In some examples, consumables are shipped in packages, boxes, etc. containing multiple units of a consumable (e.g., a box of ten needles, a bottle of two gallons of a fluid, etc.), such that step 310 includes rounding up to a nearest package size to determine a number of packages to deliver in step 310.

In some examples, the provider computing system 102 and/or the user device processing circuit 126 (or user client application 128) is configured to provide step 310 by generating an order based on the comparison between the forecasted future demand and the estimated remaining supply, optionally adjusting the order based on user input (as described in further detail below with respect to FIGS. 5-8), and submit the order to an order fulfilment system, for example via an application programming interface. Based on such an order, the order fulfillment system can cause the consumables indicated in the order to be collected, packaged, shipped, and delivered to the location of the patient and the dialysis machine 10. The provider computing system 102 and/or the user device 104 can thereby cause delivery of the quantity of new units of the consumable to the dialysis machine. The dialysis system 100 thereby provides new units of a consumable to the dialysis machine 10 in step 310.

At step 312, the dialysis machine 10 performs dialysis on the patient using one or more of the new units of the consumable delivered to the dialysis machine 10 in step 310. Step 312 can be executed during a time period in which the dialysis machine 10 would have run out of the consumable, absent execution of steps 304-310. Process 300 thereby enables continued operation of the dialysis machine 10 to provide dialysis treatment sessions to the patient at the patient's home. Further, process 300 enables such continued operation optionally without human intervention, for example with a patient or caregiver having an option to intervene in automated execution of steps 304-310 (e.g., to adjust order amounts, to make special orders, etc.) but without requiring the patient or caregiver to intervene, thereby preserving values in both ease-of-use, automation, and customizability. Step 312 can be omitted in some examples.

Referring now to FIG. 4, an illustration of a process 400 for determining quantities of consumables to deliver to a dialysis machine is shown, according to some examples. The process 400 can be used in steps 306-310 of process 300 for example. The process 400 can be executed by the provider computing system 102, the user device 104, the dialysis machine 10, or some combination thereof, in various examples.

Process 400 starts with an initial inventory 402. The initial inventory 402 indicates starting amounts of consumables, for example amounts of consumables initially provided to a patient at the time of delivery and setup of a dialysis machine 10 at the patient's home. The initial inventory 402 can be determined from data on a first order, delivery, etc. to the patient and/or a manual count of consumables located at the patient's home (e.g., by the patient or a caregiver and entered into the user device 104 using input/output circuit 134 and an interface provided by user client application 128). The initial inventory 402 thus provides a starting point for consumable inventory calculations, estimations, etc. in process 400.

As shown in FIG. 4, an order history 404 is combined with the initial inventory 402 at node 406. In particular, order history 404 includes numbers of consumables ordered for and delivered to a dialysis machine 10 over time, which is added to the initial inventory 402 at node 406 to obtain total numbers of consumables available to the dialysis machine 10 over time. A total value for each different consumable (e.g., a number of needles, a number of catheters, a volume of dialysate, etc.) can be provided as an output of node 406.

Process 400 then illustrates that a historical consumption estimate 408 is then subtracted from the output of node 406, i.e., from the total numbers of consumables available to the dialysis machine 10 over time, at node 410. The historical consumption estimate 408 as shown in FIG. 400 includes estimates of total consumption of consumables from a starting point in time associated with the initial inventory 402 to a present time (i.e., a time of execution of process 400). By subtracting such estimates of historical consumption from the output of node 406, an estimated current inventory is determined as an output of node 410.

Process 400 can thus be characterized as maintaining an up-to-date estimated inventory of consumables at a dialysis machine 10 based on an initial inventory, order history, and consumption estimates. In some examples, the output of node 410 is stored in the consumables inventory 114 of provider database 109.

Process 400 further illustrates that consumption forecasts 412 are then subtracted from the up-to-date estimated inventory (output from node 410) at node 414. The consumption forecasts 412 are amounts of consumables forecast to be consumed over an upcoming time period (e.g., over an upcoming month, over a user-selectable amount of time), for example from a current time to a next delivery date and from the next delivery date to a subsequent delivery date. The result of subtracting the consumption forecasts 412 from the up-to-date estimated inventory at node 414 is an estimate of what the consumable inventory would be at the end of the upcoming time period if no additional orders/deliveries are made. In some scenarios contemplated herein, the output of node 414 will be negative, representing a hypothetical consumables deficit at the end of the upcoming time period if no additional consumables are delivered.

As illustrated in FIG. 4, the output of node 414 is then subtracted from target inventory levels 416. The target inventory levels 416 represent target inventory amounts at the end of an upcoming time period. For example, in some scenarios a goal is to maintain a slight excess of consumables to enable continued treatment in case of delivery delays or the like, such that the target inventory levels 416 are at the end of an upcoming time period are contemplated to be non-zero. In some examples, the target inventory levels are based on the consumption forecasts 412 (e.g., half of the consumption forecasts 412 for an upcoming time period, etc.). The output of node 414 is therefore estimates of numbers of consumables (e.g., a number of units of each consumable) which should be delivered to the dialysis machine 10 in order to achieve the target inventory levels 416.

FIG. 4 further illustrates that package sizes 420 are taken into account. Different consumables 12 may be packaged for delivery in packages of different sizes (counts, volumes, etc.) as provided by manufacturers, distributors, etc. of such consumables. For example, a first consumable may come in packaging having a first number of units of the first consumable (e.g., a box of ten needles), and a second consumable may come in packaging having a second number of units of the second consumable (e.g., a bag of one hundred sanitizing wipes). Such package sizes 420 are used at node 421 where the output of node 418 (i.e., the numbers of units of each consumable estimated for delivery to achieve the target inventory levels 416) is rounded up to the nearest integer numbers of packages to determine resupply quantities 422. As one example, if an output of node 418 is that twenty-five needles are estimated to be needed to reach a target inventory level for needles, and needles come in boxes of ten needles, node 421 would use the package size 420 to determine that the resupply quantity 422 for needles is three boxes. Process 400 thereby provides resupply quantities in integer numbers of packages of each type of consumable.

The resupply quantities 422 can then be delivered to the dialysis machine 10, such that suitable numbers of consumables are available at the dialysis machine 10 over an upcoming time period (for example following approval or non-intervention by the patient or a caregiver as discussed with reference to FIGS. 5-9 below). Process 400 can be regularly repeated and updated, for example at each delivery period (e.g., monthly, at user-selectable delivery intervals) in order to repeatedly maintain suitable inventories for the dialysis machine 10 over time.

In some examples, the initial inventory 402, the order history 404, the consumption forecasts 412, and the target inventory levels 416 are quantified in number of units of each consumable, independent of package sizes, with only the resupply quantities 422 (e.g., recommend order amounts) being expressed in terms of numbers of packages. Accordingly, process 400 is able to handle mismatches between numbers of consumables used over certain time periods (e.g., in month-long periods, periods in set delivery schedule such as a user-adjustable delivery schedule) and packages sizes.

To provide an example scenario, if a dialysis machine 10 consumes six units of a consumable each month, and the consumable comes in boxes having eight units, the approach of process 400 will determine that an inventory of the consumable will grow each month if packages of eight units are continued to be delivered each month. In such an example, after several months of this mismatch, the process 400 will automatically determine that sufficient units have already been delivered to cover an upcoming time period (e.g., a zero or negative value out of node 418), such that another package of that consumable need not be included in a next delivery. Over-shipment and waste of consumables can thus be avoided by execution of process 400.

With respect to FIG. 4, the present disclosure contemplates that any equivalent or substantially equivalent mathematical formulations and orders of operations are within the scope of the present disclosure. For example, order histories 404 for many orders can be added first and then compared to a sum of many corresponding historical consumption estimates 408, net values for multiple subperiods can be calculated first by comparing order histories 404 and consumption estimates 408 for each subperiod before rolling up to a present value, or running, iterative inventory levels can be kept and updated with iterations of process 400. All such implementations and variations thereof are within the scope of the present disclosure.

FIG. 4 further illustrates that dialysis machine operating data 424, dialysis machine settings 426, a treatment plan 428, and the order history 404 can be inputs used to determine the historical consumption estimates 408 and the consumption forecasts 412. Various rules-based approaches, look-up tables, analytical functions, regression models, machine-learning models, artificial intelligence tools, etc. can be used in various examples to provide the historical consumption estimates 408 and the consumption forecasts 412 based on some combination of the order history 404, the dialysis machine operating data 424, dialysis machine settings 426, and/or the treatment plan 428.

The dialysis machine operating data 424 is operating data provided by the dialysis machine 10 as described elsewhere herein. The operating data 424 can indicate a number of treatments performed in a historical time period, a duration of such treatments, consumables identified during such treatments, etc., which can be useful in generating historical consumption estimates 408. For example, in some examples an assumption is made that a certain number of units of a consumable (e.g., half, one, two) are used per treatment session, such that the number of treatments indicated in the operating data 424 can determine the historical consumption estimates 408 (e.g., by multiplying the number of treatment sessions by the number of units per treatment session). Such information can also be used to generate consumption forecasts 412, for example by estimating that future consumption will have similarities to historical consumption.

The dialysis machine settings 426 are settings for the dialysis machine 10, for example settings for a historical period (which can be used for historical consumption estimates 408) or settings (or changes to settings) that can be used for consumption forecasts 412. A treatment plan 428, including a treatment plan for a historical period and/or for a future period can also be used. For example, a treatment plan 428 can indicate a prescribed number of treatment sessions and a duration of such sessions that a patient is instructed to perform by a caregiver, which can be used with a per-treatment consumption rate to generate consumption forecasts 412 and/or historical consumption estimates 408. In some examples, the treatment plan 428 is user-input (e.g., by a caregiver) via user device 104 or an interface of the dialysis machine 10.

The order history 404 can also be used in generating historical consumption estimates 408 and consumption forecasts 412. For example, the order history 404 may reflect that a particular patient repeatedly ordered a particular amount of a consumable, which may be more than a standard or expected amount, for example by repeatedly increasing or decreasing a recommended amount output of the consumable determined in previous iterations of process 400. Such data may indicate that the patient's particular use of the dialysis machine 10 has historically consumed more of the consumable than an expected amount (e.g., more than an amount than indicated in typical instructions/training, affecting historical consumption estimates 408), and that the patient's behavior is likely to continue such consumption, thereby affecting the consumption forecasts 412. Various artificial intelligence (AI)/machine learning (ML) tools, trend recognition algorithms, etc. can be used to implement such features. For example, in some implementations, AI/ML approaches such as neural networks/deep neural networks, reinforcement learning, Bayesian analyses, etc. can be utilized. In some examples, such models can be trained on consumption data, order data, operating data, etc. associated with other dialysis machines, for example such that one or more models learn to estimate or predict consumption amounts based on operating data inputs, treatment plans, or the like.

Referring now to FIGS. 5-8, a sequence of interface views displayed on user device 104 are shown, according to various examples. In particular, FIGS. 5-8 illustrates the user device 104 providing an application (e.g., user client application 128) which enables a user (e.g., a patient, a caregiver) to review and modify orders and deliveries of consumables, for example in coordination with execution of process 300 and/or 400.

As shown FIG. 5, the user device 104 displays a graphical user interface 500. The graphical user interface 500 indicates a date for a next scheduled delivery of consumables 12 to the dialysis machine 10, and includes a prompt 502 indicating that the user has until a certain date to modify the consumables and quantities thereof that will be provided in the shipment (e.g., according to a set shipment schedule, according to a user-adjustable shipment schedule, according to a caregiver-adjustable shipment schedule, etc.). In some examples, such consumables and quantities are auto-populated according to process 300 and/or 400, and will be delivered in the auto-populated amounts unless edited by the user. The graphical user interface 500 is also shown as including a warning 504 that the user is estimated as having a low inventory of one or more consumables 12.

By selection of prompt 502, the user device 104 can navigate to a view where consumables planned for delivery to the dialysis machine 10 are shown, for example the view in the graphical user interface 500 shown in FIG. 6. In the example shown, the consumables planned for delivery include acid concentrate, heparin, and hemodialysis dialyzers, indicated by a first bar 600 for the acid concentrate, a second bar 602 for the heparin, and a third bar 604 for the hemodialysis dialyzer. The first bar 600, the second bar 602, and the third bar 604 indicate planned amounts of each that will be shipped (e.g., four cases of acid concentrate, one case of heparin, two boxes of hemodialysis dialyzer), for example amounts output from process 400 (i.e., the resupply quantities 422). In some examples, the graphical user interface 500 can be scrolled downwards to show planned amounts for more consumables.

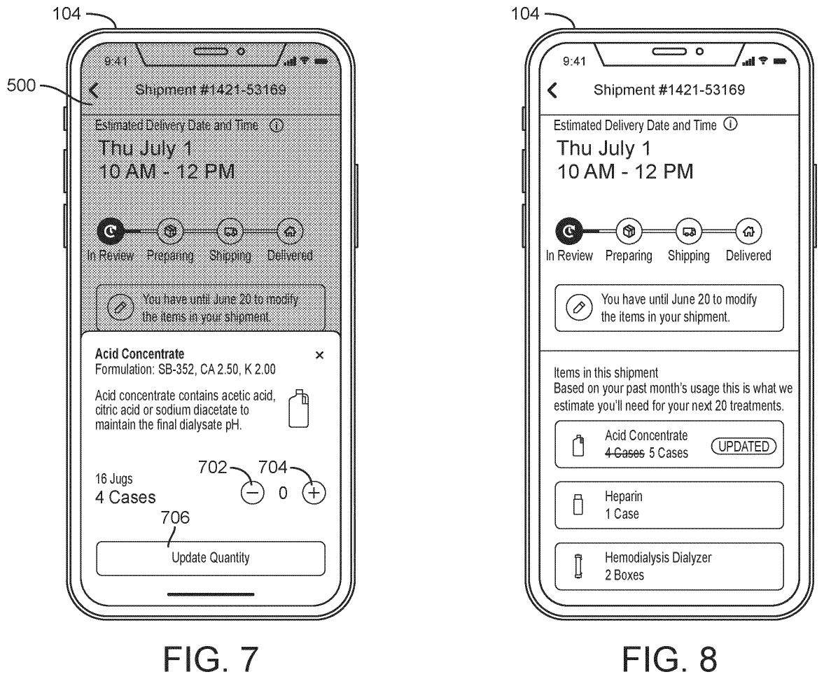

By selection of one of the first bar 600, the second bar 602, and the third bar 604, the user can navigate to a view in which the quantity of the corresponding consumable to be delivered can be edited by the user. FIG. 7 shows a view in the graphical user interface 500 reached by selecting the first bar 600, which is configured to enable a user to edit the number of cases of acid concentrate that will be delivered. As shown in FIG. 7, the graphical user interface 500 includes a minus button 700 selectable to reduce the quantity of the consumable to be delivered and a plus button 702 selectable to increase the quantity of the consumable to be delivered. If the minus button 700 or the plus button 702 are selected, the graphical user interface 500 will update to reflect the change.

In some examples, a constraint is placed on the user's ability to adjust the quantity of the consumable to be delivered. For example, the user client application 128 may be configured to prevent the user from increasing the quantity to be delivered by more than a threshold amount (e.g., by more than 50%) or to above a threshold quantity. The user client application 128 may also or alternatively be configured to prevent the user from decreasing the quantity to be delivered by more than a threshold amount (e.g., by more than 50%) or to below a threshold quantity. Such constraints can be set by a patient's physician (e.g., nephrologist), a nurse, other staff at a dialysis treatment center, according to standard of care information, etc. In some examples, the user client application 128 includes a feature which enables a user to send a prompt or initiate a call to an appropriate approver (e.g., physician, nurse, other professional, etc.) to override such a constraint.

When a user has adjusted an amount using the minus button 700 or the plus button 702, the graphical user interface 500 can update to allow selection of an update button 706. Selection of the update button 706 causes the change in quantity of the consumable to be saved and implemented, so that the user-selected amount will be delivered in the next delivery.

Further, selection of the update button 706 can cause the graphical user interface 500 to navigate to the view shown in FIG. 8. In the example of FIG. 8, a user selected to increase a number of units of acid concentrate to be included in a next delivery. The graphical user interface 500 shows the first bar 600, the second bar 602, and the third bar 604, now with the first bar 600 updated to include an icon 606 indicating that the quantity was updated by the user. The first bar 600 may also show both the recommend quantity (e.g., with a strikethrough) and the updated, user-selected quantity.

FIGS. 5-8 thus illustrate that the teachings herein enable a user to adjust recommended quantities (e.g., recommendations from process 400) before such recommend quantities are delivered to the dialysis machine 10. FIGS. 5-8 also illustrate that the user need not independently order and take any action to receive appropriate amounts of consumables. That is, in the example shown, the user has not updated the amount of heparin (shown in the second bar 602) or hemodialysis dialyzer (shown in the third bar 604), such that the recommend amounts will be delivered without user intervention. The teachings herein thus provide a human-on-the-loop automation system in which the user can intervene when desired while automated actions occur without requiring human interaction, for example when a user determines that automated actions are proceeding in a desired manner. User-friendly systems and methods for ensuring that a dialysis machine 10 has continued, reliable availability of consumables 12 are thereby provided by the teachings herein.

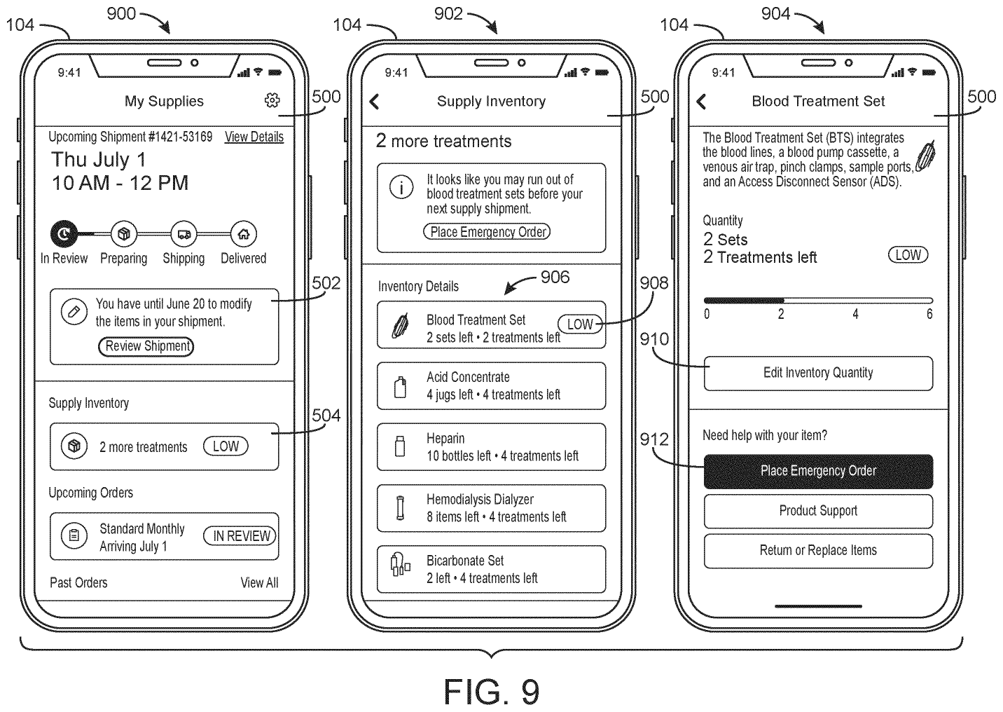

Referring now to FIG. 9, another series of views in the user interface 500 presented on the user device 104 is shown, according to some examples. FIG. 9 shows progression through a first view 900, a second view 902, and the third view 904 in the user interface 500. The first view 900 is substantially as shown in FIG. 5, including the prompt 502 and the warning 504. The warning indicates that the dialysis system 100 (e.g., the user client application 128, the provider computing system 102, the consumables inventory 114, etc. in various examples) has determined (e.g., using teachings from process 400) that a remaining inventory for one or more consumables is at a low level. In some examples, the warning 504 is generated in response to a determination that an estimated remaining inventory is less than a forecast consumption from a current time to the next delivery date, i.e., an estimation that a dialysis machine 10 will run out of a consumable before the next scheduled delivery date. The warning 504 can indicate a number of treatments estimated by the dialysis system 100 to be left before a supply is exhausted (e.g., before insufficient consumables are left to provide further treatment).

By selection of the warning 504, the graphical user interface 500 is caused to navigate to the second view 902. In the second view 902, the graphical user interface 500 provides further information about the warning 504. Further, the second view 902 includes an inventory 906 showing a list of consumables, the estimated remaining quantity of each consumable, and an estimated number of treatments believed to be enabled by the remaining quantity of each consumable. The inventory 906 includes a low icon 908 indicating a consumable for which a low (e.g., insufficient) amounts is estimated to be remaining at the dialysis machine 10. In the example shown, a low number of blood treatment sets are estimated to be remaining (estimated to be not yet consumed).

By selection of one an entry on the inventory 906, the graphical user interface 500 is caused to navigate to the third view 904. In the third view 904, the graphical user interface 500 prompts the user to take action to resolve the warning 504. The graphical user interface 500 is shown as including an edit inventory quantity button 910, which is selectable to allow the user to manually input an actual inventory amount to override and reset an estimate generated by the dialysis system 100. For example, automated estimates according to process 400 over-estimated consumption, a higher quantity of a consumable may remain than is estimated. The edit inventory quantity button allows the user to count the number of the consumable available at the dialysis machine 10 and directly input that number into the user device 104, which may resolve the warning depending on the number input. Such user input can then be used as the initial inventory 402 in process 400.

The third view 904 also shows the graphical user interface 500 as including a place emergency order button 912. Selection of the place emergency order button 912 can cause the user client application 128 to order, with rush delivery parameters, a sufficient amount of the consumable expected to resolve the warning (e.g., to last until the next scheduled supply delivery). The third view 904 is also show as including buttons to enable a user to access product support information or return or replace consumables.

The hardware and data processing components used to implement the various processes, operations, illustrative logics, logical blocks, modules and circuits described in connection with the examples disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some examples, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary example, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The examples of the present disclosure may be implemented using existing computer processors, or by a special purpose computer processor for an appropriate system, incorporated for this or another purpose, or by a hardwired system. Examples within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

A number of implementations have been described. Nevertheless, it will be understood that additional modifications may be made without departing from the scope of the inventive concepts described herein, and, accordingly, other examples are within the scope of the following claims.

What is claimed is:

1. A dialysis system, comprising:
a dialysis machine configured to perform dialysis for a patient while causing consumption of a consumable; and
circuitry programmed to automatically cause additional units of the consumable to be provided to the dialysis machine in a quantity determined, by the circuitry, based on estimated historical consumption of the consumable and an estimated future consumption of the consumable.

2. The dialysis system of claim 1, wherein:
the dialysis machine is configured to provide, to the circuitry, operating data relating to performing the dialysis; and
the circuitry is programmed to determine the estimated historical consumption of the consumable based on the operating data.

3. The dialysis system of claim 2, wherein the operating data comprises an amount of the consumable measured by the dialysis machine or an indication that the consumable was consumed in performing the dialysis for the patient.

4. The dialysis system of claim 2, wherein the operating data comprises a count of dialysis treatments provided by the dialysis machine.

5. The dialysis system of claim 4, wherein the circuitry is programmed to determine the estimated historical consumption of the consumable further based on a treatment plan for the patient.

6. The dialysis system of claim 1, wherein:
the dialysis machine is configured to perform the dialysis in accordance with a setting for the dialysis machine; and
the circuitry is programmed to forecast the estimated future consumption of the consumable based on the setting for the dialysis machine.

7. The dialysis system of claim 1, wherein the circuitry is programmed to determine the quantity further based on an order history for the consumable.

8. The dialysis system of claim 1, wherein the circuitry is further programmed to provide a graphical user interface comprising an option for a user to adjust the quantity.

9. A method of automatically providing supplies for home dialysis treatment with a dialysis machine, comprising:
receiving, from the dialysis machine, operating data indicating consumption of a first unit of a consumable in conjunction with the home dialysis treatment;
estimating a remaining amount of the consumable based on the operating data;
forecasting future demand for the consumable based on the operating data; and
causing a quantity of new units of the consumable to be delivered to a location of the dialysis machine based on a comparison of the forecasted future demand and the estimated remaining amount to be used for subsequent home dialysis treatments.

10. The method of claim 9, further comprising providing, by the dialysis machine, the home dialysis treatment to a patient using new units of the consumable.

11. The method of claim 9, wherein estimating the remaining amount of the consumable based on the operating data comprises:
determining, from the operating data, a number of treatment sessions provided by the dialysis machine; and
estimating a historical consumption of the consumable based on the number of treatment sessions.

12. The method of claim 9, wherein forecasting future demand for the consumable based on the operating data is based on a setting for the dialysis machine, the setting associated with a treatment plan for the home dialysis treatment.

13. The method of claim 9, further comprising providing a user interface comprising an indication of the remaining amount of the consumable or the quantity of new units of the consumable.

14. The method of claim 12, further comprising allowing a user to adjust the quantity of new units of the consumable via the user interface.

15. The method of claim 9, wherein estimating the remaining amount of the consumable is further based on an order history for the consumable.

16. Non-transitory computer-readable media storing program instructions that, when executed by the one or more processors, cause the one or more processors to perform operations comprising:
receiving, from a dialysis machine, operating data indicating consumption of a consumable in conjunction with a home dialysis treatment;
estimating a remaining amount of the consumable based on the operating data;
forecasting future demand for the consumable based on the operating data; and
causing a quantity of new units of the consumable to be delivered to a location of the dialysis machine based on a comparison of the forecasted future demand and the estimated remaining amount to be used for subsequent home dialysis treatments.

17. The non-transitory computer-readable media of claim 16, wherein the operations further comprise providing a user interface comprising an indication of the remaining amount of the consumable or the quantity of new units of the consumable.

18. The non-transitory computer-readable media of claim 17, the operations comprising enabling a user to adjust the quantity of new units of the consumable via the user interface.

19. The non-transitory computer-readable media of claim 16, wherein estimating the remaining amount of the consumable is further based on an order history for the consumable.

20. The non-transitory computer-readable media of claim 16 wherein forecasting future demand for the consumable based on the operating data is based on a setting for the dialysis machine, the setting associated with a treatment plan for the home dialysis treatment.
